(19) United States
(12) Patent Application Publication
(10) Pub. No.: US 2006/0258736 A1
(43) Pub. Date: Nov. 16, 2006

(54) DOSING REGIMEN

(75) Inventor: Maurizio Voi, Stamford, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company, Princeton, NJ (US)

(21) Appl. No.: 11/382,729

(22) Filed: May 11, 2006

Related U.S. Application Data
(60) Provisional application No. 60/680,398, filed on May 12, 2005.

(51) Int. Cl. A61K 31/337

(52) U.S. Cl. 514/449

(57) ABSTRACT

This invention relates to a twice weekly administration of 3′-tert-Butyl-3′-N-tert-butyloxycarbonyl-4-deacetyl-3′-dephenyl-3′-N-debenzoyl-4-O-methoxycarbonyl-paclitaxel that has been found to reduce the neurotoxicity of the drug and allow for better patient compliance in the use of this oral cancer drug.

### RELATED APPLICATION

This application claims the benefit of U.S. Provisional Application No. 60/680,698, filed on May 13, 2005, incorporated herein by reference in its entirety.

### FIELD OF THE INVENTION

This invention relates to a twice weekly administration of 3′-tert-butyl-3′-N-tert-butyloxycarbonyl-4-deacetyl-3′-dephenyl-3′-N-debenzoyl-4-O-methoxycarbonyl-paclitaxel that has been found to reduce the neurotoxicity of the drug and allow for better patient compliance in the use of this oral cancer drug.

### BACKGROUND OF THE INVENTION

U.S. Pat. No. 6,750,246 describes C-4 methyl carbonate taxane analogs which have been shown to possess surprising oral activity and thus would have utility against proliferative diseases after oral administration. WO 03/053350 discloses pharmaceutical compositions of orally effective taxane derivatives and their use for inhibiting tumor growth in mammalian hosts. The entire disclosures of each of the aforementioned patents and patent publications are incorporated herein by reference.

A particularly advantageous C-4 methyl carbonate taxane analog that has been found to have superior oral activity is 3′-tert-butyl-3′-N-tert-butyloxycarbonyl-4-deacetyl-3′-dephenyl-3 ′-N-debenzoyl-4-O-methoxycarbonyl-paclitaxel, having the structure of formula I:

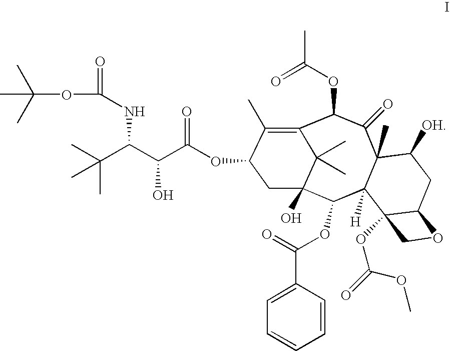

A patent application disclosing particular crystalline forms used in the dosing regiment and methods of the invention was filed November 22, 2005 as U.S. Ser. No. 11/285,463.

In accordance with the present invention, it has been shown in a Phase I clinical trial that a twice weekly administration of 3′-tert-butyl-3′-N-tert-butyloxycarbonyl-4-deacetyl-3′-dephenyl-3′-N-debenzoyl-4-O-methoxycarbonyl-paclitaxel to patients with advanced solid tumors may reduce the neurotoxicity of the drug.

Due to the anticipated oral dose of the drug (200 mg/m2), a weekly treatment regimen requires a large number of capsules to be swallowed at one time. Even with a very large 25 mg capsule, 8 pills are required which can lead to problems with patient compliance. The twice weekly treatment regimen of the invention cuts the dosage in half resulting in better patient compliance.

### SUMMARY OF THE INVENTION

This invention relates to a twice weekly dosage regimen for the administration of 3′-tert-Butyl-3′-N-tert-butyloxycarbonyl-4-deacetyl-3′-dephenyl-3′-N-debenzoyl-4-O-methoxycarbonyl-paclitaxel.

In particular, it has been found that this dosing regimen reduces peripheral neuropathy which was the main and dose limiting toxicity on a weekly treatment regimen.

The invention also provides a method of treating cancer by administering the compound on a twice weekly treatment regimen. In particular, the invention provides a method for treating solid tumor types such as non-small cell lung cancer (NSCLC) or prostate cancer.

Additionally, the invention provides a method for treating cancer in a patient that has histologically confirmed, advanced solid tumors refractory to standard therapy.

Other aspects of the invention will be apparent from the detailed description of the invention.

### DETAILED DESCRIPTION OF THE INVENTION

In accordance with the present invention, it has been shown in a Phase I clinical trial that a twice weekly administration of 3′-tert-butyl-3′-N-tert-butyloxycarbonyl-4-deacetyl-3′-dephenyl-3′-N-debenzoyl-4-O-methoxycarbonyl-paclitaxel to patients with advanced solid tumors may reduce the neurotoxicity of the drug.

The following are definitions of terms that are used in the present specification. The initial definition provided for a group or term herein applies to that group or term throughout the present specification individually or as part of another group, unless otherwise indicated.

The term “twice weekly dosing regimen” means a continuous dosing schedule with drug given, for example, on Mondays and Thursday or Tuesdays and Fridays.

The term “ECOG performance status 0-2” is defined as shown below

ECOG Performance Status
These scales and criteria are used by doctors and researchers to assess how
a patient's disease is progressing, assess how the disease affects the
daily living abilities of the patient, and determine appropriate treatment
and prognosis. They are included here for health care professionals to access.
ECOG PERFORMANCE STATUS*

| Grade | ECOG |
|---|---|
| 0 | Fully active, able to carry on all pre-disease performance without restriction |
| 1 | Restricted in physically strenuous activity but ambulatory and able to carry |
| | out work of a light or sedentary nature, e.g., light house work, office work |
| 2 | Ambulatory and capable of all selfcare but unable to carry out any work |
| | activities. Up and about more than 50% of waking hours |
| 3 | Capable of only limited selfcare, confined to bed or chair more than 50% of |
| | waking hours |
| 4 | Completely disabled. Cannot carry on any selfcare. Totally confined to bed or |
| | chair |
| 5 | Dead |

*As published in Am. J. Clin. Oncol.:
Oken, M. M., Creech, R. H., Tormey, D. C., Horton, J., Davis, T. E., McFadden, E. T., Carbone, P. P.: Toxicity And Response Criteria Of The Eastern Cooperative Oncology Group. Am J Clin Oncol 5: 649-655, 1982.

The compounds of formula I may form salts which are also within the scope of this invention. Pharmaceutically acceptable (i.e. non-toxic, physiologically acceptable) salts are preferred, although other salts are also useful, e.g., in isolating or purifying the compounds of this invention.

The compounds of formula I may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds for formula I may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art.

The dosing regimen and methods of the invention are useful in the treatment of a variety of cancers, including (but not limited to) the following:
carcinoma, including that of the bladder, breast, colon, kidney, liver, lung, including small cell lung cancer, non-small cell lung cancer, esophagus, gall bladder, ovary, pancreas, stomach, cervix, thyroid, prostate, and skin, including squamous cell carcinoma;
hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkitt's lymphoma;
hematopoietic tumors of myeloid lineage, including acute and chronic myelogenous leukemias, myelodysplastic syndrome and promyelocytic leukemia;
tumors of mesenchymal origin, including fibrosarcoma and rhabdomyosarcoma;
tumors of the central and peripheral nervous system, including astrocytoma, neuroblastoma, glioma and schwannomas; and
other tumors, including melanoma, seminoma, teratocarcinoma, osteosarcoma, xenoderoma pigmentosum, keratoctanthoma, thyroid follicular cancer and Kaposi's sarcoma.

### Materials and Methods

### Compound

3′-tert-Butyl-3′-N-tert-butyloxycarbonyl-4-deacetyl-3′-dephenyl-3′-N-debenzoyl-4-O-methoxycarbonyl-paclitaxel was synthesized by Bristol-Myers Squibb (BMS) chemists. The compound was dissolved initially in equal portions of Cremophor EL and ethanol, followed by aqueous dilution to yield final ethanol and Cremophor EL concentrations of 10%.

The compounds are administered in a 5 mg capsule formulation composed of the following ingredients:

| Ingredient | Amount | | Percent |
|---|---|---|---|
| Compound I | 5.0 | mg | 4.0% |
| Polyethylene Glycol | 104.875 | mg | 83.9% |
| 3350, NF | | | |
| Tween 80, NF | 15.0 | mg | 12.0% |
| Citric Acid Anhydrous, | 0.125 | mg | 0.1% |
| USP/EP | | | |

Alternatively, the compound is administered in a 20 or 25 mg mg capsule containing the same ingredients in proportion to the 5 mg capsule. All capsules are hard gelatin. The 5 mg capsules are size #1 and the 25 mg capsules used are size #0.

### Methods

Patients with histologically confirmed, advanced solid tumors refractory to standard therapy, ECOG performance status 0-2, and adequate live, renal and bone marrow function without symptomatic brain metastasis or pre-existing neuropathy ≧grade 2 were eligible for the trial.

The starting dose was 60 mg/m² with planned dose escalation of 20 mg/m² for each next cohort, consisting of at least 3 patients. Cohorts were expanded to 6 patients upon occurrence of a dose limiting toxicity (DLT). No premedication was required.

Toxicity was evaluated according to NCI-CTC (version 2.0).

Dose limiting toxicities (DLTs) were defined as any of the following drug related events occurring during the first cycle (first 4 weeks): grade 4 neutropenia>5 days, febrile neutropenia, grade 4 thrombocytopenia or grade 3 requiring platelet transfusion, any grade 3-4 non-hematological toxicity, QTc interval>500 msec, any significant arrhythmia, any dose reduction or omission due to toxicity.

Tumor response was evaluated according to the modified WHO criteria.

### Results

A total of 19 patients have been enrolled between January 2004 and February 2005 as shown below in Table 1.

TABLE 1
Patient Characteristics

| Characteristics | No. |
|---|---|
| Total No. of Patients | 19 |
| Male | 17 |
| Female | 2 |
| Age (years) | |
| Median | 63 |
| Range | 49-73 |
| ECOG Performance status | |
| 0 | 3 |
| 1 | 14 |
| 2 | 2 |
| Prior Chemotherapy | |
| 1 Regimen | 7 |
| 2 Regimen | 7 |
| 3 Regimen | 4 |
| Histology | |
| NSCLC | 7 |
| Colon | 3 |
| Prostate | 3 |
| Esophagus | 2 |
| Other | 4 |

The patients have been treated at escalating dose levels of 60 (n=3), 80 (n=10) and 100 mg/M² (n=6). Table 2 summarizes the side effects occurring during any cycle considered possibly, probably or certainly related to Compound I. One patient at 80 mg/ m² had to be replaced. No dose limiting toxicities (DLTs) have been observed in the patients at 60 mg/m². At the 80 mg/m² dose level, 2 DLTs were observed consisting of a grade 3 fatigue and a dose-omission due to grade 2 peripheral neuropathy. At the 100 mg/m² dose level, no DLTs were observed in the first three patients but one patient developed grade 4 neutropenia, grade 3 fatigue and grade 3 hematuria shortly after the first cycle. Although this did not meet the criteria set for a DLT, the cohort was expanded to 6 patients for safety reasons.

TABLE 2
Drug-related side effects (any cycle) in 19 patients (60-100 mg/m²)

| | Gr 1-2 | Gr 3 | Gr 4 |
|---|---|---|---|
| Hematological | | | |
| Neutropenia | 1 | | 1 |
| Thrombocytopenia | 5 | | |
| Neurology | | | |
| Sens. neuropathy | 10 | | |
| Motor neuropathy | 1 | | |
| Gastrointestinal | | | |
| Nausea | 3 | | |
| Vomiting | 2 | | |
| Anorexia | 6 | | |
| Stomatitis | 1 | | |
| Diarrhea | 9 | | |
| Taste disturbance | 7 | | |
| Pain | | | |
| Myalgia | 3 | | |
| Abdominal pain | 2 | | |
| Other | | | |
| Alopecia | 3 | | |
| Fatigue | 7 | 2 | |
| Epistaxis | 7 | | |
| Hematuria | | 1 | |

As shown in Table 3 below, neuropathy appears to be less frequent and less severe on the twice weekly treatment regimen of the invention as compared to a weekly schedule.

TABLE 3
Neuropathy (any cycle)

| | Weekly schedule (n = 48) | Twice weekly schedule (n = 19) |
|---|---|---|
| Grade 1 | 6 (13%) | 8 (42%) |
| Grade 2 | 19 (40%) | 2 (10%) |
| Grade 3 | 6 (12%) | |
| Grade 4 | | |
| Total (%) | 31 (65%) | 10 (52%) |

With regard to efficacy, three confirmed partial responses were observed in 18 response-evaluable patients. This was seen in 2 of 6 response-evaluable NSCLC patients and 1 of 3 prostate carcinoma patients.

In conclusion, Compound I is well tolerated when given on a twice weekly treatment regimen. Neuropathy appears to occur less frequently and to be less severe than on a weekly treatment schedule.

Promising activity has been observed in patients with NSCLC and prostate carcinoma indicating that Compound I is active on a twice weekly treatment regimen.

We claim:

1. A method of administering 3′-tert-butyl-3′-N-tert-butyloxycarbonyl-4-deacetyl-3′-dephenyl-3′-N-debenzoyl-4-O-methoxycarbonyl-paclitaxel or a pharmacologically acceptable salt thereof of formula (I) 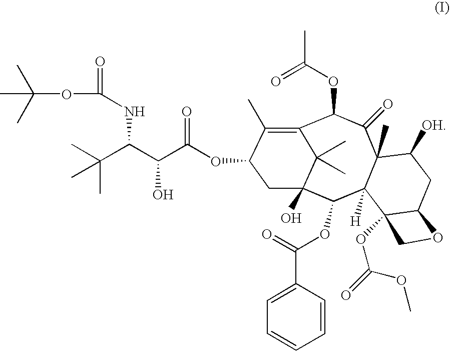
to a patient in need thereof, said method comprising orally administering a dose of about 60 mg/m² to 100 mg/m² in a twice weekly treatment regimen.

2. The method according to claim 1 wherein the compound of formula (I) is administered to a patient being treated for cancer.

3. The method according to claim 2 wherein the patient is being treated for solid tumor types including non-small cell lung cancer and prostate cancer.

4. The method according to claim 3 wherein the patient being treated has histologically confirmed, advanced solid tumors refractory to standard therapy.

5. A method for treating non-small cell lung cancer comprising administering 3′-tert-butyl-3′-N-tert-butyloxycarbonyl-4-deacetyl-3′-dephenyl-3′-N-debenzoyl-4-O-methoxycarbonyl-paclitaxel or a pharmacologically acceptable salt thereof of formula (I) 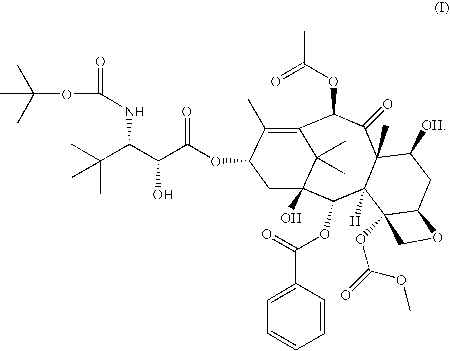
at a dose of about 60 mg/m2 to 100 mg/m2 in a twice weekly treatment regimen to a patient in need thereof.

6. A method for treating prostate cancer comprising administering 3′-tert-butyl-3′-N-tert-butyloxycarbonyl-4-deacetyl-3′-dephenyl-3′-N-debenzoyl-4-O-methoxycarbonyl-paclitaxel or a pharmacologically acceptable salt thereof of formula (I) 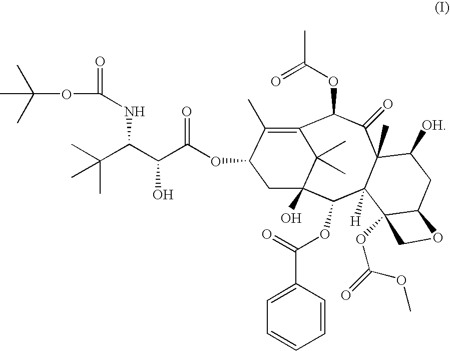
at a dose of about 60 mg/m2 to 100 mg/m2 in a twice weekly treatment regimen to a patient in need thereof.

7. The method according to claim 5 wherein the compound is administered orally.

8. The method according to claim 6 wherein the compound is administered orally.

9. A method for reducing neuropathy versus a weekly treatment schedule by administering 3′-tert-butyl-3′-N-tert-butyloxycarbonyl-4-deacetyl-3′-dephenyl-3′-N-debenzoyl-4-O-methoxycarbonyl-paclitaxel or a pharmacologically acceptable salt thereof of formula (I) 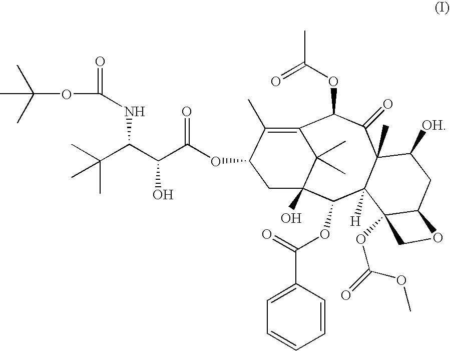
to a patient in need thereof on a twice weekly treatment schedule.

10. The method according to claim 9 wherein the patient is being treated for cancer.

11. The method according to claim 10 wherein the cancer for which the patient is being treated is advanced solid tumors.

12. The method according to claim 11 wherein the advanced solid tumors for which the patient is being treated are those associated with non-small cell lung cancer or prostate cancer.
